(19) United States
(12) Patent Application Publication
(10) Pub. No.: US 2026/0284345 A1
(43) Pub. Date: Sep. 24, 2026

(54) LARYNGEAL MASK AIRWAY

(71) Applicant: Zhejiang UE Medical Corp., Taizhou (CN)

(72) Inventors: Jiazhi ZHANG, Taizhou (CN); Kairi HUANG, Taizhou (CN); Rui FAN, Taizhou (CN); Weidong WANG, Taizhou (CN); Taohong WANG, Taizhou (CN)

(73) Assignee: Zhejiang UE Medical Corp., Taizhou (CN)

(21) Appl. No.: 19/193,748

(22) Filed: Apr. 29, 2025

(30) Foreign Application Priority Data
Mar. 18, 2025 (CN) 202520470519X

(51) Int. Cl. A61M 16/06; A61B 1/05; A61B 1/267

(52) U.S. Cl. CPC A61M 16/0605; A61B 1/05; A61B 1/267; A61M 2202/0007; A61M 2202/02; A61M 2205/583; A61M 2209/10; A61M 2210/065

(57) ABSTRACT

The present application discloses a laryngeal mask airway, and relates to the technical field of medical equipment. The laryngeal mask airway comprises: a mask bag, an air duct and an inflation tube; the air duct comprises a joint and an air passage, wherein the joint is used for connecting a gas delivery device; one end of the air passage is connected with the joint, and the other end is connected with the mask bag; the air duct is provided with an opening; one end of the inflation tube is connected with the opening, and the other end is connected with an inflation port on the mask bag, wherein when the gas delivery device delivers gas to a throat of a patient through the air duct, the gas inflates the mask bag through the inflation tube to form sealing between the mask bag and the throat.

### CROSS-REFERENCE TO RELATED APPLICATIONS

This application claims priority to Chinese Patent Application No. 202520470519.X, filed on Mar. 18, 2025, the content of which is incorporated herein by reference in its entirety.

### TECHNICAL FIELD

The present application relates to the technical field of medical equipment, in particular to a laryngeal mask airway.

### BACKGROUND

The laryngeal mask airway (LMA) is a piece of medical equipment for respiratory tract management, and is mainly used for quickly establishing an artificial airway in the scenarios of clinical anesthesia, first aid, intensive care, etc. to keep the respiratory tract of a patient unobstructed and prevent aspiration at the same time. An airbag laryngeal mask airway is a frequently-used target laryngeal mask airway.

The traditional airbag laryngeal mask airway is an inflatable laryngeal mask airway. After the laryngeal mask airway is put into the throat of the patient by medical staff, an airbag is manually inflated to form sealing between the inflated airbag and the throat structure of the patient, thereby preventing oxygen inputted into the throat of the patient from leaking.

However, this traditional laryngeal mask airway requires manual inflation of the airbag. The establishment speed of the artificial airway may be affected by the step process of the inflation operation.

### SUMMARY

In view of this, embodiments of the present application provide a laryngeal mask airway, which does not require manual inflation, eliminates the operation process of manual inflation and achieves the rapid establishment of an artificial airway.

Embodiments of the present application provide a laryngeal mask airway, comprising: a mask bag, an air duct and an inflation tube; the air duct comprises a joint and an air passage, wherein the joint is used for connecting a gas delivery device; one end of the air passage is connected with the joint, and the other end is connected with the mask bag; the air duct is provided with an opening; one end of the inflation tube is connected with the opening, and the other end is connected with an inflation port on the mask bag, wherein when the gas delivery device delivers gas to a throat of a patient through the air duct, the gas inflates the mask bag through the inflation tube to form sealing between the mask bag and the throat.

In one possible embodiment, the shape and the structure of the mask bag after inflation and the shape and the structure of the air duct are adapted to those of the throat.

In one possible embodiment, the inflation tube and the air duct are integrated within separate chambers, or the inflation tube is embedded in the air duct.

In one possible embodiment, the opening is arranged on the joint.

In one possible embodiment, a ratio of the tube diameter of the inflation tube to the tube diameter of the air passage is within a preset range which is determined according to set inflation time and the set air pressure of the mask bag.

In one possible embodiment, the laryngeal mask airway further comprises an observation cavity, and the observation cavity is embedded into the air duct or the observation cavity is composed of an additional duct;

The observation cavity comprises an observation window which is made of transparent material; a groove is formed on one side of the mask bag; and the observation window is located on the groove side of the mask bag.

In one possible embodiment, the laryngeal mask airway further comprises a camera and a visual apparatus;

The camera is installed in conjunction with the observation cavity, the camera is connected with the visual apparatus, and a connection line therebetween is arranged inside the observation cavity.

In one possible embodiment, the laryngeal mask airway further comprises a cleaning tube; one port of the cleaning tube is arranged in the groove and installed in conjunction with the observation window to clean the observation window; and another port of the cleaning tube is connected with a cleaning joint, wherein the cleaning joint is used for connecting and inputting a cleaning solution.

In one possible embodiment, the cleaning tube is nested inside the air duct, or arranged integrally with the air duct in separate cavities.

In one possible embodiment, the laryngeal mask airway further comprises a drainage tube; one port of the drainage tube is arranged in the groove, and one port is used for connecting a gastric tube; and another port of the drainage tube is fixed to one end of the air duct not inserted into the throat.

In one possible embodiment, the drainage tube is nested inside the air duct, or arranged integrally with the air duct in separate cavities.

The present application has the following beneficial effects:

The laryngeal mask airway provided by the embodiments of the present application uses the inflation tube connected with the air duct to automatically inflate the mask bag by the gas in the process of gas delivery of the air duct. Compared with the traditional technical solution of manual inflation, this technical solution has the following advantages:
(1) Automatic inflation and simplification of the operation process. The traditional manual inflatable laryngeal mask airway needs to be manually inflated by medical staff after the laryngeal mask airway is inserted. The operation is cumbersome and it is easy to lead to poor sealing due to insufficient or excessive inflation. The laryngeal mask airway of the present application is automatically inflated in the process of gas delivery without manual operation, thereby reducing the operation steps and time of the medical staff and increasing efficiency.
(2) More ideal sealing effect. When the traditional laryngeal mask airway is manually inflated, there may be the situation that the inflation pressure is insufficient or an indication airbag is misleading, resulting in incomplete sealing. The laryngeal mask airway in the technical solution of the present application is directly inflated through the gas delivery device, which can control the inflation pressure more precisely, ensure the formation of good sealing between the mask bag and the throat, and reduce the risk of air leakage.
(3) Improvement of the safety of airway management. An automatic inflation mechanism in the technical solution of the present application reduces the problem of airway sealing caused by inaccuracy of manual inflation, reduces the risk of aspiration due to regurgitation, and is particularly suitable for anesthesia and first-aid scenarios.

Other features and advantages of the present application will be illustrated in the following description, and become apparent in part from the description, or will be known through the implementation of the present application. The purpose and other advantages of the present application can be realized and obtained through the structures specially pointed out in the written description, claims and drawings.

### BRIEF DESCRIPTION OF DRAWINGS

To more clearly describe the technical solutions in the embodiments of the present application or in the related technologies, the drawings required to be used in the description of the embodiments or the related technologies will be simply presented below. Apparently, the drawings in the following description are merely the embodiments of the present application, and for those ordinary skilled in the art, other drawings can also be obtained according to the provided drawings without contributing creative labor.

FIG. 1 is a structural schematic diagram 1 of a laryngeal mask airway in an embodiment of the present application;

FIG. 2 is a sectional view of an air passage and an inflation tube in an embodiment of the present application;

FIG. 3 is a structural schematic diagram of a display device in an embodiment of the present application;

FIG. 4 is a structural schematic diagram 2 of a laryngeal mask airway in an embodiment of the present application;

FIG. 5 is a structural schematic diagram 3 of a laryngeal mask airway in an embodiment of the present application;

FIG. 6 is a structural schematic diagram 4 of a laryngeal mask airway in an embodiment of the present application.

Mask bag—101; air duct—102; inflation tube—103; observation cavity—301; camera—401; visual apparatus—402; image output apparatus—403; adapter cable—404; cleaning tube—501; groove—1011; joint—1021; air passage—1022; one port—5011 of cleaning tube; another port—5012 of cleaning tube; drainage tube—601; one port—6011 of drainage tube; another port—6012 of drainage tube.

### DETAILED DESCRIPTION OF THE EMBODIMENTS

To make the purpose, the technical solutions and the advantages of the present application more clear, the technical solutions in the embodiments of the present application will be clearly and fully described below in combination with the drawings in the embodiments of the present application. Apparently, the described embodiments are merely part of the embodiments of the present application, not all of the embodiments. Based on the embodiments in the present application, all other embodiments obtained by those ordinary skilled in the art without contributing creative labor will belong to the protection scope of the present application. If there is no conflict, the embodiments in the present application and features in the embodiments can be mutually combined at random. Moreover, although logical sequences are shown in the flow charts, in some cases, the steps shown or described can be executed in sequences different from the sequences here.

The terms such as “first”, “second”, etc. in the description, claims and the above drawings in the present application are used for distinguishing similar objects, rather than used for describing special sequences or precedence orders. It shall be understood that such data used may be exchanged under appropriate circumstances so that the embodiments of the utility model described herein may be implemented in the order other than those illustrated or described herein.

The laryngeal mask airway (LMA) is a piece of medical equipment for respiratory tract management, and is mainly used for quickly establishing an artificial airway in the scenarios of clinical anesthesia, first aid, intensive care, etc. to keep the respiratory tract of a patient unobstructed and prevent aspiration at the same time. An airbag laryngeal mask airway is a frequently-used target laryngeal mask airway. The traditional airbag laryngeal mask airway is an inflatable laryngeal mask airway. After the laryngeal mask airway is put into the throat of the patient by medical staff, an airbag is manually inflated to form sealing between the inflated airbag and the throat structure of the patient, thereby preventing oxygen inputted into the throat of the patient from leaking. However, this traditional laryngeal mask airway requires manual inflation of the airbag. The establishment speed of the artificial airway may be affected by the step process of the inflation operation.

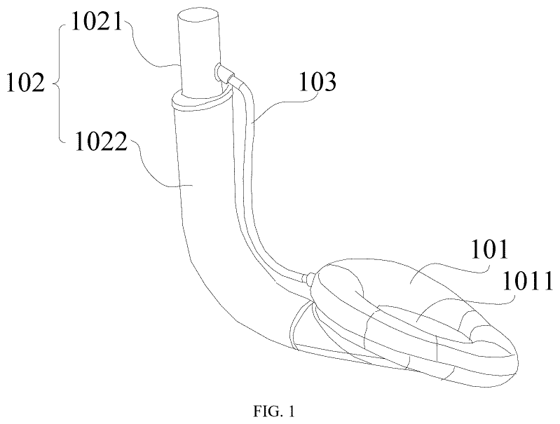

In view of this, as shown in FIG. 1, an embodiment of the present application provides a laryngeal mask airway which comprises: a mask bag 101, an air duct 102 and an inflation tube 103; the air duct 102 comprises a joint 1021 and an air passage 1022, wherein the joint 1021 is used for connecting a gas delivery device; one end of the air passage 1022 is connected with the joint 1021, and the other end is connected with the mask bag 101; one surface of the mask bag 101 is concave inward to form a groove 1011, and the groove 1011 is connected with one end of the air passage 1022; the air duct 102 is provided with an opening; one end of the inflation tube 103 is connected with the opening, and the other end of the inflation tube 103 is connected with an inflation port on the mask bag, wherein when the gas delivery device delivers gas to a throat of a patient through the air duct 102, the gas inflates the mask bag 101 through the inflation tube 103 to form sealing between the mask bag 101 and the throat.

In the present embodiment, the inflation tube 103 and the air duct 102 are arranged separately, and a protecting tube body is arranged outside the air duct 102. The shapes and the structures of the mask bag 101 after inflation and the air duct 102 are adapted to the shape and the structure of the throat. Before the laryngeal mask airway is put into a human body, the mask bag is deflated through manual air extraction, and then the laryngeal mask airway is put into the airway. When oxygen is inputted into the throat of the patient through the gas delivery device such as an oxygen generator, an ambient air vaporizer, etc. by the air duct 102, the inputted oxygen is diverted to the mask bag 101 through the inflation tube 103 or the inputted oxygen preferably flows to the mask bag 101 through a diversion switch, to form a gas communication channel of the airbag—the inflation tube—the air duct, so as to inflate the mask bag 101. The shape and the structure of the mask bag after inflation are adapted to the shape and the structure of the throat of the patient to closely adhere to the throat, so as to quickly establish the artificial airway.

Optionally, an interface of the inflation tube 103 on the air duct 102 is located on the joint 1021 of the air duct 102.

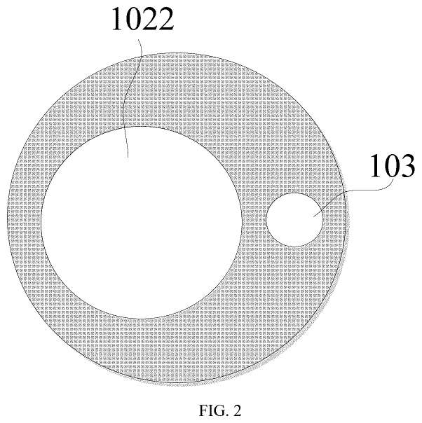

In one embodiment, the inflation tube 103 and the air duct 102 are integrated within separate chambers, as shown in FIG. 2 which is a sectional view of the arrangement of the inflation tube 103 and the air passage 1022. In FIG. 2, a large circular hole is the section of the air passage 1022, and a small circular hole is the section of the inflation tube 103. Compared with arrangement in separate tubes, this integrated arrangement in separate cavities can make the overall structure of the laryngeal mask airway more compact and reduce the operation steps and time. Meanwhile, this mode can also better adapt to the anatomical structure of the throat and ensure closer fitting between the laryngeal mask airway and the throat, thereby enhancing the sealing performance.

In one embodiment, the inflation tube 103 is embedded in the air duct 102. Thus, the overall volume and the complexity of the laryngeal mask airway are reduced, making the insertion and the operation of the laryngeal mask airway simpler. Meanwhile, this design avoids the problem of pipeline entanglement or misoperation that may occur when the inflation tube and the air duct are arranged separately in the traditional laryngeal mask airway. In addition, the embedded design reduces the exposed part of the inflation tube, and reduces the risk that the inflation tube is bitten by the teeth of the patient or clamped by an air duct fixer, thereby avoiding ventilation failure caused by blockage or accidental displacement of the inflation tube. This design is suitable for patients who need long-term indwelling of the laryngeal mask airway and can significantly reduce the occurrence of related complications.

In one embodiment, a ratio of the tube diameter of the inflation tube 103 to the tube diameter of the air passage 1022 is within a preset range which is determined according to set inflation time and the set air pressure of the mask bag. The inflation time determines the speed at which the gas flows into the mask bag 101 from the inflation tube 103. If the inflation time is too long, the rapid inflation of the laryngeal mask airway may be affected; and if the inflation time is too short, excessively high requirements may be proposed for the flow rate and the pressure of the inflation tube 103. In addition, the mask bag 101 needs to be kept sealed at the set air pressure while avoiding compression on the throat tissues. The set air pressure is generally determined according to clinical requirements and safety. In actual design, the ratio of the tube diameters can be adjusted through simulation and experiments to ensure that the inflation tube can inflate the mask bag quickly and stably under the set inflation time and air pressure.

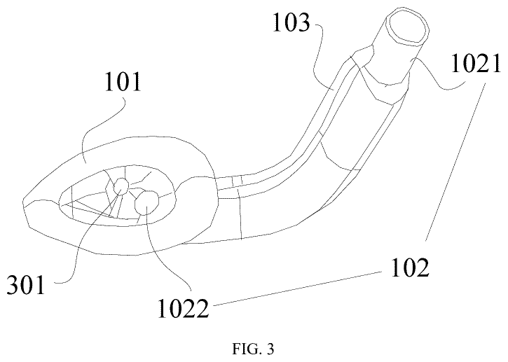

In one embodiment, as shown in FIG. 3, the laryngeal mask airway further comprises an observation cavity 301, and the observation cavity 301 is embedded into the air duct 102. An observation window of the observation cavity 301 is made of transparent material generally selected from material with high transparency, good biocompatibility and mechanical strength, such as polycarbonate or acrylic resin. In addition, the observation window 301 is located on the groove side of the mask bag 101 so that medical staff can directly observe the condition of the throat for facilitating precise positioning and adjustment in the process of inserting the laryngeal mask airway. Meanwhile, this layout reduces the interference with the throat tissues, and improves the comfort and the safety of use. The image data of the throat can also be recorded in combination with an endoscope or other imaging devices through the observation window 301, which is convenient for subsequent analysis and diagnosis. In addition, the medical staff can find abnormal conditions, such as edema or bleeding, of the throat in time through the observation window 301, thereby taking corresponding measures to reduce postoperative complications.

Optionally, the observation cavity 301 is composed of an additional duct.

In one embodiment, the surface of the observation window 301 is provided with an anti-fogging layer to avoid generating fog spots due to temperature changes or humidity influences during use, so as to ensure a clear field of vision. The size and the shape of the observation window 301 are designed according to actual needs, which should not only meet the observation need, but also minimize the influence on the overall structure of the laryngeal mask airway.

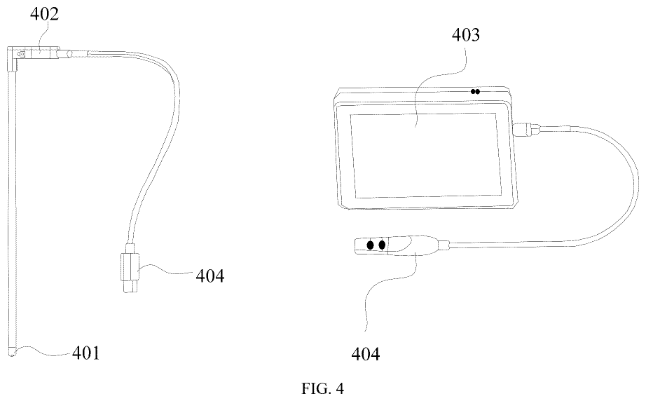

In one embodiment, as shown in FIG. 4 which is a structural schematic diagram of a display device, the display device can be a part of the laryngeal mask airway and is used in conjunction with the laryngeal mask airway. The display device comprises a camera 401 and a visual apparatus 402. The camera 401 is installed in conjunction with the observation cavity 301. The camera 401 is installed near the observation cavity 301 and used in conjunction with the transparent observation window, which can capture the image data of the throat in real time. The camera 401 is connected with the visual apparatus 402. A connection line therebetween is arranged in the observation cavity 301. The internal structure of the observation cavity 403 is optimized, which can effectively reduce signal interference and ensure the clarity and the stability of image transmission. The visual apparatus 402 can transmit images captured by the camera 401 to an external display or mobile device in real time. The medical staff can intuitively observe the condition of the throat through these devices to ensure the accurate placement of the laryngeal mask airway. In addition, the visual apparatus 402 also supports an image recording function, which is convenient for postoperative analysis and teaching.

In one embodiment, the observation cavity 301 is composed of an additional duct other than the air duct.

In one embodiment, the display device further comprises an image output apparatus 403, the visual apparatus 402 is also connected with the image output apparatus 403 through an adapter cable 404, and the condition of the throat can be intuitively displayed through the image output apparatus 403.

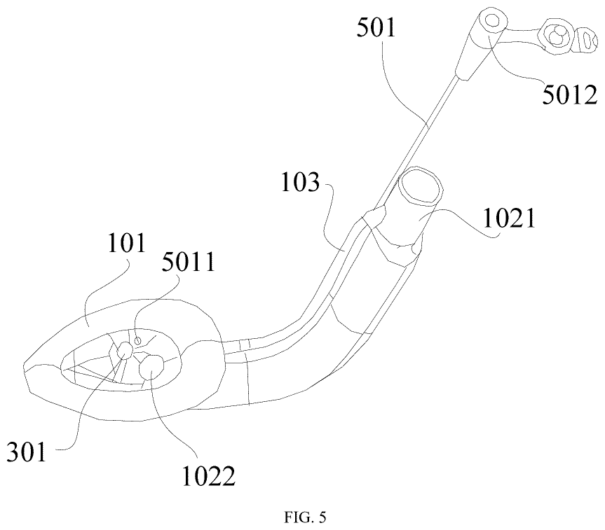

In one embodiment, as shown in FIG. 5, the laryngeal mask airway further comprises a cleaning tube 501; one port 5011 of the cleaning tube 501 is arranged in the groove and installed in conjunction with the observation window 301 to clean the observation window 301; and another port 5012 of the cleaning tube 501 is connected with a cleaning joint, wherein the cleaning joint is used for connecting and inputting a cleaning solution.

Specifically, the cleaning tube 501 is an important part of the laryngeal mask airway, and one port 5011 of the cleaning tube is arranged in the groove of the mask bag 101 and installed in conjunction with the observation window 301 to clean the observation window 301. This ensures that the observation window can be always kept clear during use, which avoids affecting the field of vision due to secretions or stains. In addition, another port 5012 of the cleaning tube 501 is connected with the cleaning joint for connecting the cleaning solution, thereby achieving the immediate cleaning of the observation window. In some designs, the cleaning tube 501 can also be connected with a recovery system to recover the used cleaning solution, to avoid pollution.

In addition, the material selection of the cleaning tube 501 needs to meet the requirements of biocompatibility and durability to ensure that performance is maintained during frequent use and cleaning. For example, corrosion-resistant and wear-resistant medical-grade material can be used to extend the service life.

In one embodiment, by referring to FIG. 5, the cleaning tube 501 is nested inside the air duct 102.

In one embodiment, the cleaning tube 501 and the air duct 102 are formed by being integrated within separate chambers.

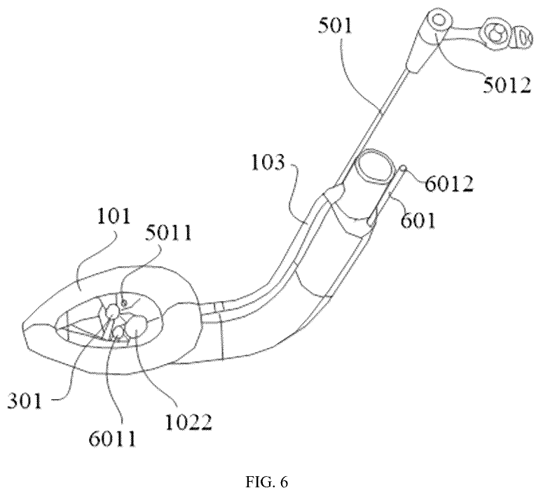

In one embodiment, as shown in FIG. 6, the laryngeal mask airway further comprises a drainage tube; one port 6011 of the drainage tube 601 is arranged in the groove for connecting a gastric tube; another port 6012 of the drainage tube 601 is fixed to one end of the air duct 102 not inserted into the throat; the fixation mode here is assembly type fixation, which is detachable for facilitating cleaning; and of course, the drainage tube can also be integrally formed, which is not specifically limited here. Wherein the port 6011 of the drainage tube is connected with the gastric tube, which can effectively drain gas and liquid in the gastrointestinal tract, reduce the pressure on the gastrointestinal tract, and prevent the reflux and the aspiration of gastric contents. Preferably, a reflux prevention mechanism, such as a check valve or a plug assembly, is arranged in the drainage tube 601 to prevent drainage fluid from flowing back into an esophagus or the throat and reduce the risk of aspiration.

In one embodiment, by referring to FIG. 6, the drainage tube 601 is nested inside the air duct 102.

In one embodiment, the drainage tube 601 and the air duct 102 are integrated within separate chambers.

Structural components shown in the above embodiments can be combined with each other.

Obviously, those skilled in the art can implement various modifications and variations for the present application without departing from the spirit and the scope of the present application. So, the present application is intended to include these modifications and variations if these modifications and variations of the present application belong to claims of the present application and the equivalent technical scope.

What is claimed is:

1. A laryngeal mask airway, comprising: a mask bag, an air duct and an inflation tube;
the air duct comprises a joint and an air passage, wherein the joint is used for connecting a gas delivery device;
one end of the air passage is connected with the joint, and the other end is connected with the mask bag, wherein
the air duct is provided with an opening; one end of the inflation tube is connected with the opening, and the other end is connected with an inflation port on the mask bag,
wherein when the gas delivery device delivers gas to a throat of a patient through the air duct, the gas inflates the mask bag through the inflation tube to form sealing between the mask bag and the throat.

2. The laryngeal mask airway according to claim 1, wherein the shape and the structure of the mask bag after inflation and the shape and the structure of the air duct are adapted to those of the throat.

3. The laryngeal mask airway according to claim 1, wherein the inflation tube and the air duct are integrated within separate chambers, or the inflation tube is embedded in the air duct.

4. The laryngeal mask airway according to claim 1, wherein the opening is arranged on the joint.

5. The laryngeal mask airway according to claim 1, wherein a ratio of the tube diameter of the inflation tube to the tube diameter of the air passage is within a preset range which is determined according to set inflation time and the set air pressure of the mask bag.

6. The laryngeal mask airway according to claim 1, further comprising an observation cavity, wherein the observation cavity is embedded into the air duct or the observation cavity is composed of an additional duct;
the observation cavity comprises an observation window which is made of transparent material; a groove is formed on one side of the mask bag; and the observation window is located on the groove side of the mask bag.

7. The laryngeal mask airway according to claim 6, further comprising a camera and a visual apparatus, wherein
the camera is installed in conjunction with the observation cavity, the camera is connected with the visual apparatus and a connection line therebetween is arranged inside the observation cavity.

8. The laryngeal mask airway according to claim 6, further comprising a cleaning tube, wherein
one port of the cleaning tube is arranged in the groove and installed in conjunction with the observation window to clean the observation window;
another port of the cleaning tube is connected with a cleaning joint, wherein the cleaning joint is used for connecting and inputting a cleaning solution.

9. The laryngeal mask airway according to claim 8, wherein the cleaning tube is nested inside the air duct, or arranged integrally with the air duct in separate cavities.
